(19) United States
(12) Patent Application Publication
(10) Pub. No.: US 2019/0175258 A1
(43) Pub. Date: Jun. 13, 2019

(54) TREATMENT TOOL

(71) Applicant: Olympus Corporation, Tokyo (JP)

(72) Inventor: Shoei Tsuruta, Tokyo (JP)

(73) Assignee: Olympus Corporation, Tokyo (JP)

(21) Appl. No.: 16/280,255

(22) Filed: Feb. 20, 2019

Related U.S. Application Data
(63) Continuation of application No. PCT/JP2016/078709, filed on Sep. 28, 2016.

(51) Int. Cl. A61B 18/14

(52) U.S. Cl. CPC A61B 18/1445; A61B 2018/00101; A61B 18/1482

(57) ABSTRACT

The disclosed technology is directed to a treatment tool comprises a first grasping jaw having a first grasping surface. A second grasping jaw having a second grasping surface and is configured to engage with the first grasping jaw so as to relatively pivot with respect to one another for holding a living tissue therebetween. A first electrode is disposed on the first grasping surface. A second electrode is disposed on either the first grasping surface or the second grasping surface and is configured to generate high-frequency energy in tandem with the first electrode to the living tissue held therebetween. A floating electrode is disposed in at least one of the first grasping surface and the second grasping surface. The floating electrode includes respective first and second ends each of which is disposed between the first electrode and the second electrode.

### CROSS-REFERENCE TO RELATED APPLICATION

This application is a continuation application of PCT Application No. PCT/JP2016/078709 filed on Sep. 28, 2016, which is hereby incorporated by reference in its entirety.

### TECHNICAL FIELD

The disclosed technology relates to a treatment tool.

### DESCRIPTION OF THE RELATED ART

Heretofore, there has been known a treatment tool that treats, e.g., joins or anastomoses and separates, a living tissue by grasping the living tissue with a pair of jaws and applying an energy to the living tissue, i.e., passing a high-frequency electric current through the living tissue (see, for example, Patent Literature (PTL 1)—JP 2010-527704T).

PTL 1 discloses various structures for passing a high-frequency electric current widthwise or transversely across the jaws.

According to the first structure, for example, one (hereinafter referred to as “first grasping jaw”) of the paired jaws has a first grasping surface for grasping a living tissue between itself and the other jaw (hereinafter referred to as “second grasping jaw”). The first grasping surface has a first electrode disposed on one transverse end side thereof. The second grasping jaw has a second grasping surface for grasping a living tissue between itself and the first grasping surface. The second grasping surface has a second electrode disposed on the other transverse end side thereof. In other words, the first and second electrodes are disposed in transversely staggered positions so that they do not face each other when the first and second grasping jaws are closed. When high-frequency electric power is supplied between the first and second electrodes, a high-frequency electric current flows through the living tissue grasped by the first and second grasping jaws, widthwise across the jaws.

According to the second structure, for example, the first grasping surface has a first electrode disposed on one transverse end side thereof. The first grasping surface also has a second electrode disposed on the other transverse end side thereof. When high-frequency electric power is supplied between the first and second electrodes, a high-frequency electric current flows through the living tissue grasped by the first and second grasping jaws, widthwise across the jaws.

With the structures described hereinbefore in which the high-frequency electric current flows transversely across the jaws, a region through which the high-frequency electric current flows between the first and second electrodes becomes a heat-generating region. Therefore, a treatment target tissue of the living tissue can be limited to a nearly transversely central region of the jaws between the first and second electrodes. The effect of heat on peripheral tissues that are positioned transversely outside of the jaws in the periphery of the treatment target tissue is thus reduced, allowing the living tissue to be treated minimally invasively.

A comparison will hereinafter be made between the structure disclosed in PTL 1 in which a high-frequency electric current flows widthwise across the jaws (hereinafter referred to as “width structure”) and a structure in which a high-frequency electric current flows in a direction along which the jaws face each other (hereinafter referred to as “facing structure”), unlike PTL 1. The facing structure is a structure in which the first grasping surface has a first electrode and the second grasping surface has a second electrode, so that the first and second electrodes face each other when the first and second grasping jaws are closed.

The width structure has a long electric current path along which a high-frequency electric current flows through a living tissue, compared with the facing structure. For example, when the first and second grasping jaws grasp a living tissue, the distance between the first and second grasping surfaces is 1 mm or less. Depending on the living tissue, the distance is less than 0.5 mm. In other words, the facing structure has an electric current path that is 1 mm or less long. On the other hand, it is difficult for the width structure to reduce the distance between the first and second electrodes because the treatment target tissue needs to be of a certain size. Consequently, the width structure has an electric current path that is 2 mm or 3 mm or more long.

Either the width structure or the facing structure consumes the same amount of high-frequency electric power required for treating a treatment target tissue of one kind and size. On the other hand, the electric resistance value of a living tissue, which means the real part of an electric impedance when a high-frequency electric current flows, increases in proportion to the length of the electric current path and is in inverse proportion to the cross section of the electric current path. In other words, since the length of the electric current path is larger in the width structure than in the facing structure and the cross section of the electric current path is smaller in the width structure than in the facing structure, the electric resistance value of the width structure is higher than the electric resistance value of the facing structure in treating a treatment target tissue of one kind and size.

Specifically, it is assumed that the size of a treatment target tissue is represented by a width of 3 mm, a length of 5 mm, and a thickness of 1 mm. In this case, the length of the electric current path of the facing structure is 1 mm, and the cross section thereof is 15 mm². The length of the electric current path of the width structure is 3 mm, and the cross section thereof is 5 mm². As described hereinbefore, the electric resistance value of the living tissue is in proportion to the length of the electric current path and is in inverse proportion to the cross section of the electric current path. Consequently, the electric resistance value of the width structure is nine times the electric resistance value of the facing structure. If the electric resistance value R is nine times larger, then in order to generate the same electric power P, a voltage V that is three times higher is required as can be seen from the following equation (1):

$$\begin{matrix}[ Math . 1 ] & \\ P = VI = \frac{{V}^{2}}{R} & ( 1 )\end{matrix}$$

As described hereinbefore, the width structure requires a high voltage compared with the facing structure in treating a treatment target tissue of one kind and size.

In order to reduce the voltage, it is necessary to reduce the electric resistance. However, if the distance between the first and second electrodes is simply made shorter, then the size of the treatment target tissue is reduced, possibly resulting in a failure to obtain a desired level of performance after the treatment.

Therefore, there is a need for a technology that is capable of reducing a voltage required to treat a treatment target tissue while performing the treatment minimally invasively without reducing the size of the treatment target tissue.

### BRIEF SUMMARY OF EMBODIMENTS

The disclosed technology has been made in view of the foregoing. It is an object of the disclosed technology to provide a treatment tool that is capable of reducing a voltage required to treat a treatment target tissue while performing the treatment minimally invasively without reducing the size of the treatment target tissue.

The disclosed technology is directed to a treatment system used for treatment of a body tissue by applying electrical energy thereto. The treatment system comprises a controller and a treatment tool configured to be attached to controller. The treatment tool comprises a shaft having a first end and a second end. A handle is attached to the first end. Respective first and second grasping jaws each of which having respective first and second grasping surfaces configured to be engaged with the second end of the shaft so as to pivot with respect to one another for holding living tissue therebetween during the treatment. A first electrode is disposed on the first grasping surface. A second electrode is disposed on either the first grasping surface or the second grasping surface and is configured to generate high-frequency energy in tandem with the first electrode to the living tissue being held therebetween. At least one floating electrode is disposed in at least one of the respective first and second grasping surfaces so that the treatment tool being capable of reducing a voltage required to treat the body tissue while performing the treatment without reducing a size of the body tissue.

The treatment tool according to the disclosed technology is advantageous in that it can perform a treatment minimally invasively and reduce a voltage required for the treatment.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a view illustrating a treatment tool according to Embodiment 1.

FIG. 2 is a view illustrating a grasper illustrated in FIG. 1.

FIG. 3 is a view illustrating the grasper illustrated in FIG. 1.

FIG. 4 is a view illustrating the positional relationship of first and second electrodes and a floating electrode illustrated in FIGS. 2 and 3.

FIG. 5 is a conceptual diagram illustrating the advantages of Embodiment 1.

FIG. 6 is a conceptual diagram illustrating the advantages of Embodiment 1.

FIG. 7 is a conceptual diagram illustrating the advantages of Embodiment 1.

FIG. 8 is a conceptual diagram illustrating the advantages of Embodiment 1.

FIG. 9A is a view illustrating a grasper of a treatment tool according to Embodiment 2, the view depicting a path for a high-frequency electric current in a former part of a treatment process.

FIG. 9B is a view illustrating a grasper of the treatment tool according to Embodiment 2, the view depicting a path for a high-frequency electric current in a latter part of the treatment process.

FIG. 10 is a view illustrating a grasper of a treatment tool according to Embodiment 3.

FIG. 11 is a view illustrating a floating electrode illustrated in FIG. 10.

FIG. 12A is a view depicting a path for a high-frequency electric current in a latter part of a treatment process according to Embodiment 3.

FIG. 12B is a view depicting a path for a high-frequency electric current in a latter part of the treatment process according to Embodiment 3.

FIG. 13 is a view illustrating a grasper of a treatment tool according to Embodiment 4.

FIG. 14 is a view illustrating a grasper of a treatment tool according to Embodiment 5.

FIG. 15 is a view illustrating a grasper of a treatment tool according to Embodiment 6.

FIG. 16 is a view illustrating a grasper of a treatment tool according to Embodiment 7.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

Forms by which the disclosed technology is embodied, hereinafter referred to as “embodiments,” will hereinafter be described with reference to the drawings. The embodiments to be described hereinafter should not be interpreted as limiting the disclosed technology. Identical parts are denoted by identical numeral reference in figures.

### Embodiment 1

Makeup Outline of a Treatment System

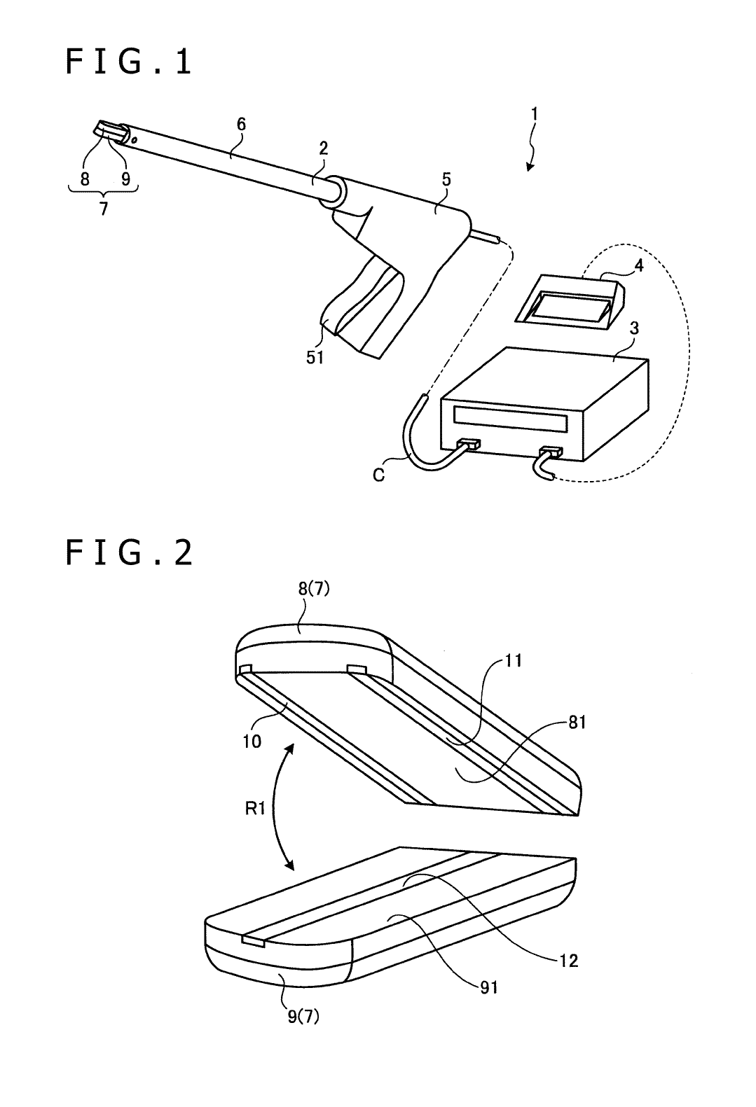

FIG. 1 is a view illustrating a treatment system 1 according to present Embodiment 1.

The treatment system 1 treats, e.g., joins or anastomoses, separates, or otherwise processes, a living tissue by applying energy, e.g., electric energy (high-frequency energy), to the living tissue. As illustrated in FIG. 1, the treatment system 1 includes a treatment tool 2, a controller 3, and a foot switch 4.

### Makeup of the Treatment Tool

The treatment tool 2 is a linear-type surgical treatment tool for treating a living tissue through an abdominal wall, for example. As illustrated in FIG. 1, the treatment tool 2 includes a handle 5, a shaft 6, and a grasper 7.

The handle 5 is a part by which the surgeon holds the treatment tool 2 by hand. As illustrated in FIG. 1, the handle 5 has a manipulating knob 51.

As illustrated in FIG. 1, the shaft 6 is of a substantially hollow cylindrical shape and has one end, i.e., a right end in FIG. 1, connected to the handle 5. The grasper 7 is mounted on the other end, i.e., a left end in FIG. 1, of the shaft 6. The shaft 6 houses therein an opening and closing mechanism, not depicted, that opens and closes a first grasping jaw 8 and a second grasping jaw 9 (FIG. 1) that make up the grasper 7 in response to the surgeon's manipulation of the manipulating knob 51. An electric cable C (FIG. 1) connected to the controller 3 is housed in the shaft 6 and extends from one end, i.e., a right end in FIG. 1, to the other end, i.e., a left end in FIG. 1, through the handle 5.

### Structure of the Grasper

“Longitudinal directions” described hereinafter refer to directions interconnecting the distal and proximal ends of the grasper 7 that is set to a closed state in which it grasps a living tissue LT, i.e., a state in which the first and second grasping jaws 8 and 9 are closed or first and second grasping surfaces 81 and 91 face each other. “Width directions” described hereinafter refer to transverse directions that extend along the first and second grasping surfaces 81 and 91 perpendicularly to the longitudinal directions.

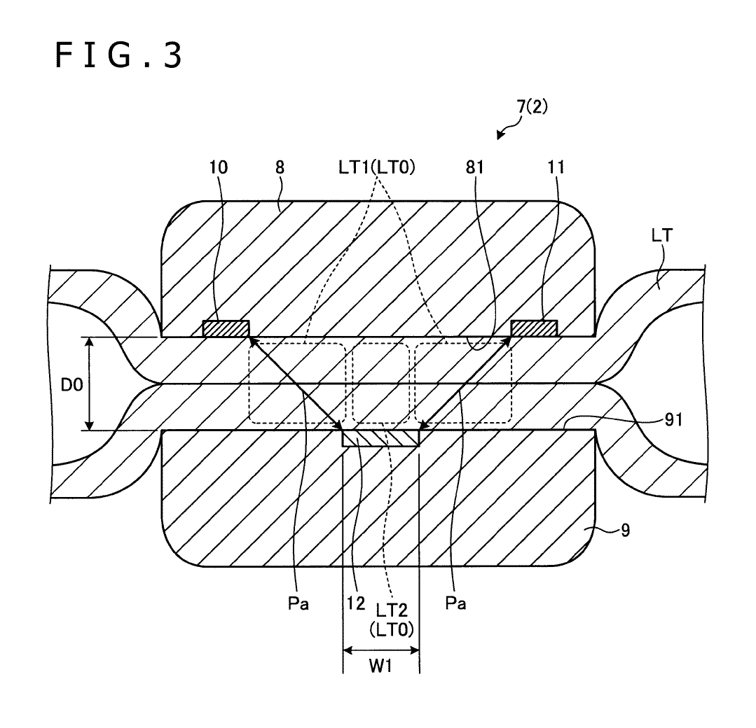

FIGS. 2 and 3 are views illustrating the grasper 7. Specifically, FIG. 2 is a perspective view illustrating the grasper 7 that is set to an open state in which the first and second grasping jaws 8 and 9 are open or spaced apart. FIG. 3 is a cross-sectional view taken along a sectional plane along the widthwise directions across the grasper 7 that is set to the closed state in which it grasps the living tissue LT such as a lumen, a blood vessel, or the like.

The grasper 7 is a portion for grasping and treating the living tissue LT (FIG. 3). As illustrated in FIGS. 1 through 3, the grasper 7 includes the first and second grasping jaws 8 and 9.

The first and second grasping jaws 8 and 9 are pivotally supported on the other end of the shaft 6 for opening and closing movement in the directions indicated by an arrow R1 (FIG. 2). The first and second grasping jaws 8 and 9 are capable of grasping the living tissue LT in response to a manipulation by the surgeon of the manipulating knob 51.

### Makeup of the First Grasping Jaw

The first grasping jaw 8 is disposed above the second grasping jaw 9 in FIGS. 2 and 3, and is substantially shaped as a rectangular parallelepiped extending along the longitudinal directions. The first grasping jaw 8 may be made of a material that is highly heat-resistant, low in thermal conductivity, and excellent in electric insulation, e.g., a resin such as PTFE (polytetrafluoroethylene), PEEK (polyetheretherketone), PBI (polybenzimidazole), or the like. However, the material of the first grasping jaw 8 is not limited to the concerned resin, but may be ceramics such as alumina, zirconia, or the like. Furthermore, the first grasping jaw 8 may be coated with PTFE, DLC (Diamond-Like Carbon), a ceramics-based insulative coating material, a silica-based insulative coating material, or a silicone-based insulative coating material that is nonadherent to living bodies.

The first grasping jaw 8 has a lower surface in FIGS. 2 and 3 that functions as a grasping surface 81 for grasping the living tissue LT between itself and the second grasping jaw 9.

According to Embodiment 1, the first grasping surface 81 has a flat shape.

As illustrated in FIGS. 2 and 3, first and second electrodes 10 and 11 are embedded in the first grasping surface 81 at respective areas positioned on both end portions in the widthwise directions or the lateral direction, i.e., on left and right end portions in FIGS. 2 and 3, and extending along the entire length, i.e., the entire length in the longitudinal directions, of the first grasping surface 81.

The first and second electrodes 10 and 11 are made of an electrically conductive material such as copper, aluminum, carbon, or the like, for example. Each of the first and second electrodes 10 and 11 is in the form of a plate substantially shaped as a rectangular parallelepiped extending along the longitudinal directions. The first and second electrodes 10 and 11 are embedded in the first grasping surface 81 such that one of the plate surfaces, i.e., the lower surface in FIGS. 2 and 3, of each of the first and second electrodes 10 and 11 makes up part of the first grasping surface 81, i.e., is exposed. The electric cable C, which extends from one end to the other end of the shaft 6, contains a pair of leads, not depicted, connected respectively to the first and second electrodes 10 and 11. When the first and second electrodes 10 and 11 are supplied with high-frequency electric power from the controller 3 through the pair of leads, the first and second electrodes 10 and 11 generate high-frequency energy. When the first and second electrodes 10 and 11 are supplied with high-frequency electric power while the first grasping jaw 8 and the second grasping jaw 9, i.e., the first grasping surface 81 and the second grasping surface 91 thereof, are grasping the living tissue LT, a high-frequency potential is developed between the first and second electrodes 10 and 11, causing a high-frequency current to flow through the living tissue LT. In other words, the first and second electrodes 10 and 11 are a pair of electrodes where one of them functions as a positive electrode while the other as a negative electrode.

The first and second electrodes 10 and 11 are not limited to plates, but may be of a different shape such as round bars embedded in the first grasping jaw 8 and having projected portions that are small as compared with the distance between the first grasping jaw 8 and the second grasping jaw 9. The first and second electrodes 10 and 11 may not necessarily be made of a bulk material, but may be in the form of electrically conductive thin films of platinum or the like deposited by way of evaporation, sputtering, or the like. Moreover, the surfaces of the first and second electrodes 10 and 11 may not necessarily be physically exposed as described hereinbefore, but may be electrically exposed. Specifically, the surfaces of the first and second electrodes 10 and 11 may be coated with an electrically conductive coating material such as Ni-PTFE film, electrically conductive DLC thin film, or the like that is nonadherent to living bodies, so that the surfaces can function as electrodes to develop a potential. Such alternatives do not depart from the scope of the disclosed technology.

### Makeup of the Second Grasping Jaw

The second grasping jaw 9 is substantially shaped as a rectangular parallelepiped extending along the longitudinal directions. As with the first grasping jaw 8, the second grasping jaw 9 may be made of a resin such as PTFE, PEEK, PBI, or the like, or ceramics such as alumina, zirconia, or the like, for example.

The second grasping jaw 9 has an upper surface in FIGS. 2 and 3 that functions as the second grasping surface 91 for grasping the living tissue LT between itself and the first grasping surface 81.

According to Embodiment 1, the second grasping surface 91 is shaped flatwise as with the first grasping surface 81.

As illustrated in FIG. 2 or 3, the second grasping surface 91 has a floating electrode 12 embedded in an area thereof that is positioned centrally in the width directions, i.e., centrally in the leftward and rightward directions in FIGS. 2 and 3, and extends the entire length of the second grasping surface 91.

The floating electrode 12 is made of a good conductor such as copper, aluminum, gold, carbon, or the like, for example. The floating electrode 12 is constructed as a plate in the form of a substantially rectangular parallelepiped extending along the longitudinal directions. The floating electrode 12 is embedded such that one plate surface thereof, i.e., an upper surface in FIGS. 2 and 3, serves as part of the second grasping surface 91, i.e., the one plate surface is exposed. Unlike the first and second electrodes 10 and 11, the floating electrode 12 is not connected to the controller 3 through a lead, and is not connected to ground, i.e., is electrically floating.

The floating electrode 12 is not limited to the shape of the plate, but may be of a different shape such as a round rod or the like embedded such that it may have a protrusive portion that is small compared with the distance between the first and second grasping jaws 8 and 9. The floating electrode 12 may not necessarily be made of a bulk material, but may be made of a foil or thin film of a good conductor or an electrically conductive DLC thin film or the like that is formed by chemical vapor deposition (CVD) or the like. The surface of the floating electrode 12 may not be physically exposed as described hereinbefore, but may be electrically exposed. In other words, the surface of the floating electrode 12 may be coated with an electrically conductive coating material such as Ni-PTFE film, electrically conductive DLC thin film, or the like which is non-adhesive to living bodies, and may provide a potential as an electrode. Such an alternative does not depart from the scope of the invention.

It is known in the art that the living tissue LT has different electric conductivities for different target regions because of different compositions thereof. For example, the volume resistivity at 10 kHz is 30 Ω·m for fat tissue, 7 Ω·m for muscle and liver tissue, and 2 Ω·m for blood. The electric conductivity differs greatly with water contents. It is also well known that the electric conductivity is quickly lost as the tissue becomes dry in the course of the treatment.

According to Embodiment 1, the floating electrode 12 has an electric resistance value of 1Ω or less, e.g., 10 mΩ, which is lower than the electric resistance value of 250Ω of the living tissue LT at the electric current path contacted by the floating electrode 12.

### Positional Relationship of the First and Second Electrodes and the Floating Electrode

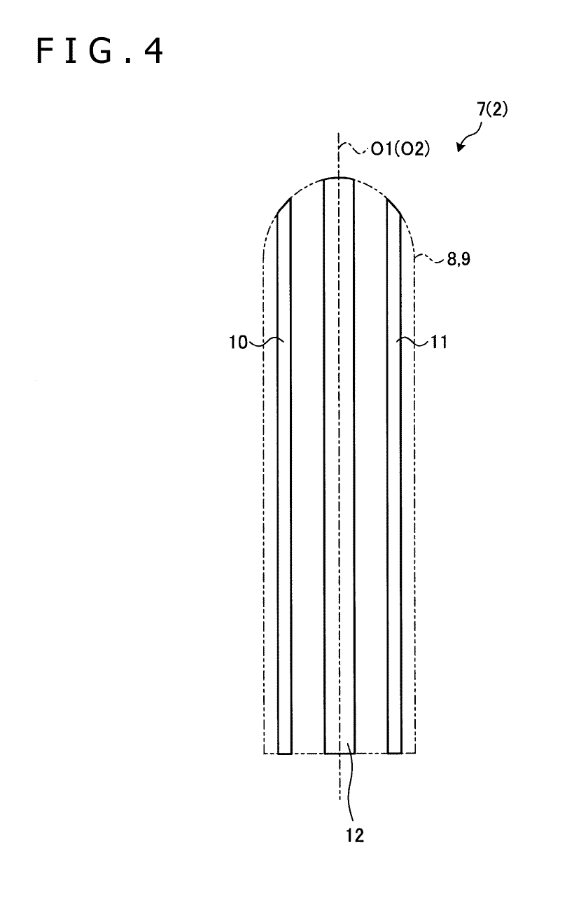

FIG. 4 is a view illustrating the positional relationship of the first and second electrodes 10 and 11 and the floating electrode 12. Specifically, FIG. 4 is a view of the first and second electrodes 10 and 11 and the floating electrode 12 as viewed along the directions in which the first and second grasping surfaces 81 and 91 face each other when the grasper 7 is in the contact state, i.e., along the directions normal to the first and second grasping surfaces 81 and 91.

As illustrated in FIG. 4, when the floating electrode 12 is viewed along the directions in which the first and second grasping surfaces 81 and 91 face each other when the grasper 7 is in the closed state, the floating electrode 12 is disposed between the first and second electrodes 10 and 11. More specifically, the floating electrode 12 has a transversely central position O1 that is aligned with a transversely central position O2 between the first and second electrodes 10 and 11.

As illustrated in FIG. 3, the floating electrode 12 has a transverse length W1 that is larger than a spaced distance D0 between the first and second grasping surfaces 81 and 91 that are grasping the living tissue LT therebetween.

### Makeup of the Controller and the Foot Switch

The foot switch 4 is a part that the surgeon operates with their foot. When the foot switch 4 is thus operated, the controller 3 selectively turns on and off the treatment tool 2, i.e., the first and second electrodes 10 and 11.

Means for selectively turning on and off the treatment tool 2 is not limited to the foot switch 4, but may be a switch that can be operated by hand, etc.

The controller 3, which includes a CPU (Central Processing Unit) and so on, integrally controls operation of the treatment tool 2 according to predetermined control programs. Specifically, in response to the operation of the foot switch 4 by the surgeon to turn on the controller 3, the controller 3 supplies high-frequency electric power at a preset output level between the first and second electrodes 10 and 11 through the pair of leads. Then, the controller 3 appropriately controls energy levels.

### Operation of the Treatment System

Next, operation of the treatment system 1 described hereinbefore will be described hereinafter.

The surgeon holds the treatment tool 2 by hand, and inserts a distal-end portion of the treatment tool 2, i.e., the grasper 7 and a portion of the shaft 6, into an abdominal cavity through the abdominal wall using a trocar or the like, for example. The surgeon also operates the manipulating knob 51 to grasp the living tissue LT with the first grasping jaw 8 and the second grasping jaw 9.

Then, the surgeon operates the foot switch 4 to turn on the controller 3 to electrically energize the treatment tool 2. When the controller 3 is turned on, the controller 3 supplies high-frequency electric power between the first and second electrodes 10 and 11 through the pair of leads.

When high-frequency electric power is supplied between the first and second electrodes 10 and 11, a high-frequency potential is generated between the first and second electrodes 10 and 11, and the floating electrode 12 is held at a potential that is substantially intermediate between the respective potentials of the first and second electrodes 10 and 11. As a result, high-frequency electric currents flow between the first and second electrodes 10 and 11 along a path that extends through only the living tissue LT and a path that extends through both the living tissue LT and the floating electrode 12. The proportions of the respective paths are determined by the difference between the electric resistance values of the living tissue LT and the floating electrode 12.

In the living tissue LT that is grasped by the first and second grasping surfaces 81 and 91, as viewed along the directions in which the first and second grasping surfaces 81 and 91 face each other, tissues positioned between the first electrode 10 and the floating electrode 12 and between the second electrode 11 and the floating electrode 12 will hereinafter be referred to as tissues LT1 (FIG. 3), and a tissue positioned between the tissues LT1 as a tissue LT2 (FIG. 3). This definition of the tissues also applies to Embodiments 2 through 6 to be described hereinafter.

According to Embodiment 1, since the floating electrode 12 is made of a good conductor, as described hereinbefore, the electric resistance value of the floating electrode 12 is far lower than the electric resistance value of the living tissue LT, or more specifically, the tissue LT2. Therefore, a high-frequency electric current flows along a path Pa that extends through the tissues LT1 and the floating electrode 12, as illustrated in FIG. 3. Thus, mainly Joule heat is generated in each of the tissues LT1. Each of the tissues LT1 is treated by the generated Joule heat. Accordingly, each of the tissues LT1 and LT2 is a treatment target tissue LT0 to be treated.

Embodiment 1 described hereinbefore offers the following advantages:

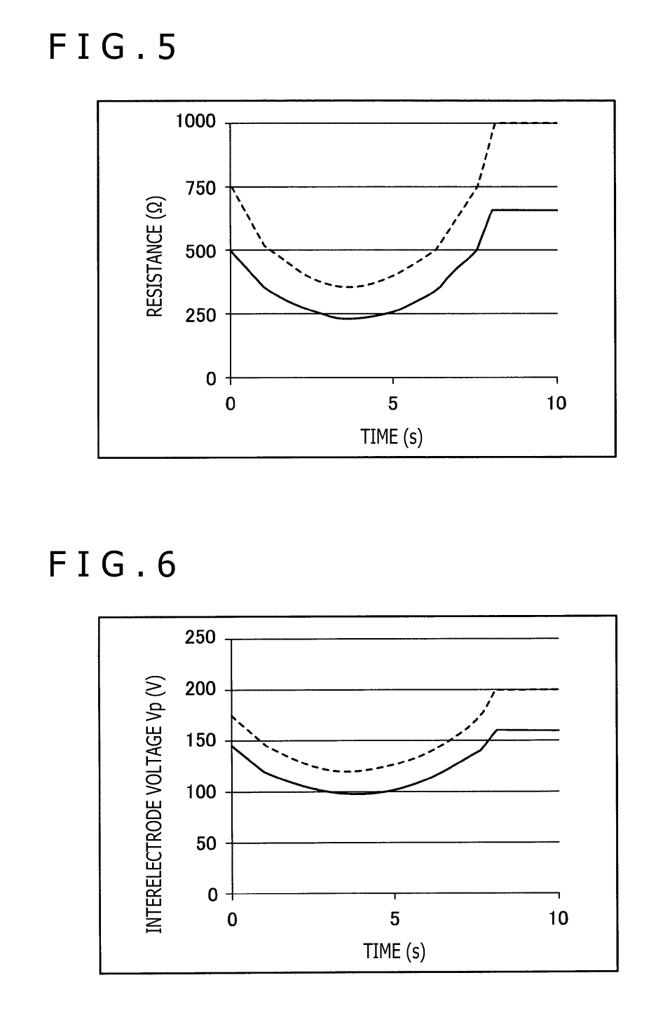

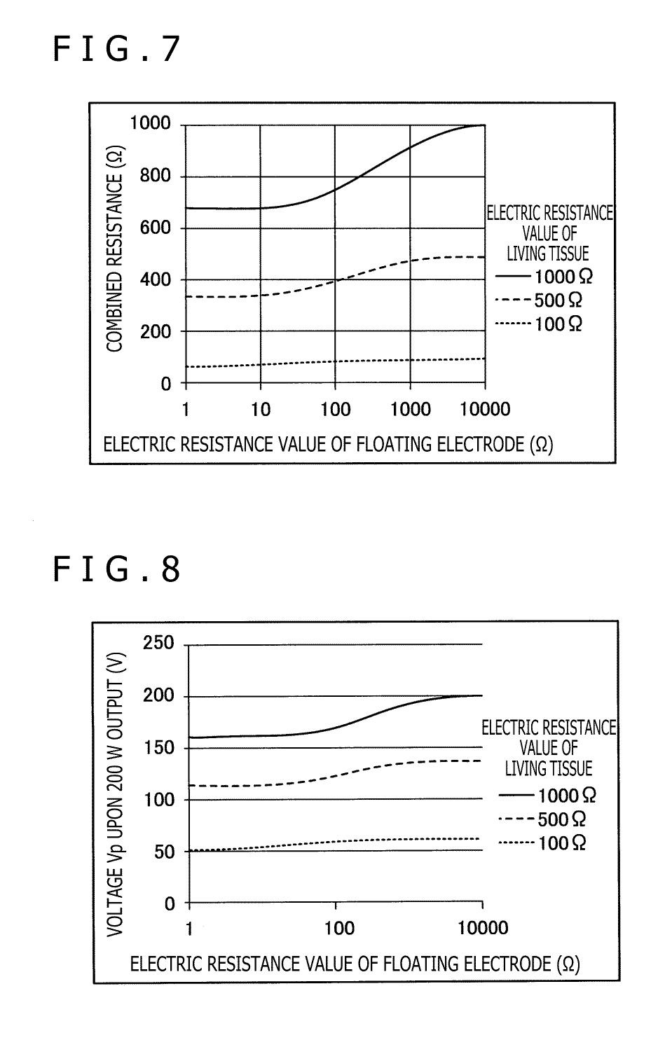

FIGS. 5 through 8 are conceptual diagrams illustrating the advantages of Embodiment 1. Specifically, FIGS. 5 and 6 illustrate, respectively, time-dependent changes in the resistance between the first and second electrodes 10 and 11 and time-dependent changes in a voltage Vp between the first and second electrodes 10 and 11 when a constant high-frequency electric power, e.g., of 20 W, is continuously supplied between the first and second electrodes 10 and 11 while the first and second grasping surfaces 81 and 91 are grasping the living tissue LT therebetween. In FIGS. 5 and 6, the time-dependent changes with the conventional structure that is free of the floating electrode 12 unlike Embodiment 1 are indicated by the broken-line curves, whereas the time-dependent changes with the structure having the floating electrode 12 according to Embodiment 1 are indicated by the solid-line curves. The solid-line curves in FIGS. 5 and 6 represent the time-depending changes with the structure having the floating electrode 12 whose electric resistance value is 1/100 of the electric resistance value of the living tissue LT, i.e., the tissue LT2, and whose length W1 is ⅓ of the distance between the first and second electrodes 10 and 11. FIGS. 7 and 8 illustrate the relationship between the electric resistance value of the floating electrode 12 and the resistance between the first and second electrodes 10 and 11, i.e., the combined resistance of the living tissue LT and the floating electrode 12, and the relationship between the electric resistance value of the floating electrode 12 and the voltage Vp between the first and second electrodes 10 and 11.

The treatment tool 2 according to Embodiment 1 includes, on the second grasping surface 91, the floating electrode 12 having the electric resistance value lower than the electric resistance value of the living tissue LT, i.e., the tissue LT2, between the first and second electrodes 10 and 11 as viewed along the directions in which the first and second grasping surfaces 81 and 91 face each other when the grasper 7 is in the closed state. Therefore, when high-frequency electric power is supplied between the first and second electrodes 10 and 11 while the first and second grasping surfaces 81 and 91 are grasping the living tissue LT therebetween, the floating electrode 12 becomes part of the path Pa of the high-frequency electric current. In other words, the floating electrode 12 is able to reduce the resistance between the first and second electrodes 10 and 11, i.e., the combined resistance of the living tissue LT and the floating electrode 12, compared with the conventional structure that is free of the floating electrode 12. The voltage required to supply predetermined high-frequency electric power between the first and second electrodes 10 and 11 can thus be made lower than with the conventional structure. Furthermore, since the voltage can be reduced simply by disposing the floating electrode 12 without reducing the distance between the first and second electrodes 10 and 11, the size of the treatment target tissue LT0 is not reduced.

Specifically, as illustrated in FIG. 5, in a latter part of the treatment process, i.e., subsequent to eight seconds in FIG. 5, the conventional structure indicated by the broken-line curve illustrated in FIG. 5 exhibits 1000Ω as the resistance between the first and second electrodes 10 and 11. On the other hand, the structure according to Embodiment 1 indicated by the solid-line curve illustrated in FIG. 5 exhibits approximately 670Ω as the combined resistance between the first and second electrodes 10 and 11, which is approximately ⅔ of the conventional structure. Accordingly, as illustrated in FIG. 6, the voltage Vp required to supply the high-frequency electric power of 20 W between the first and second electrodes 10 and 11 is 200 Vp with the conventional structure and 164 Vp with the structure according to Embodiment 1, resulting in a drop of 36 Vp.

The reduction in the combined resistance and the reduction in the voltage due to the floating electrode 12 are determined by the difference between the electric resistance values of the living tissue LT, more specifically the tissue LT2, and the floating electrode 12. Specifically, as illustrated in FIG. 7, the higher the electric resistance value of the tissue LT2 is, the larger the reduction in the combined resistance due to the floating electrode 12 becomes. As a result, as illustrated in FIG. 8, the higher the electric resistance value of the tissue LT2 is, the larger the reduction in the voltage required to supply the same high-frequency electric power between the first and second electrodes 10 and 11 becomes. Furthermore, it can be seen from FIGS. 7 and 8 that the electric resistance value of the floating electrode 12 does not need to be extremely low. For example, if the electric resistance value of the tissue LT2 is 1000Ω, then the reduction in the combined resistance and the reduction in the voltage that are caused when the electric resistance value of the floating electrode 12 is much lower than 100Ω remains essentially the same as those caused when the electric resistance value of the floating electrode 12 is 100Ω.

Moreover, the treatment tool 2 according to Embodiment 1 incorporates a width structure in which a high-frequency electric current flows widthwise across the first and second grasping jaws 8 and 9. Therefore, the treatment target tissue LT0 can be limited to a nearly transversely central region of the first and second grasping jaws 8 and 9. The effect of heat on peripheral tissues that are positioned transversely outside of the first and second grasping jaws 8 and 9 in the periphery of the treatment target tissue LT0 is thus reduced, allowing the living tissue LT to be treated minimally invasively.

In view of the foregoing, the treatment tool 2 according to Embodiment 1 is advantageous in that it is capable of reducing a voltage required to treat a treatment target tissue LT0 while performing the treatment minimally invasively without reducing the size of the treatment target tissue LT0.

With the treatment tool 2 according to Embodiment 1, furthermore, the transverse length W1 of the floating electrode 12 is larger than the spaced distance D0. Therefore, the electric resistance value of the floating electrode 12 is secured, making it possible for the floating electrode 12 to serve more reliably as part of the path Pa for the high-frequency electric current.

With the treatment tool 2 according to Embodiment 1, in addition, the transversely central position O1 of the floating electrode 12 is aligned with the transversely central position O2 between the first and second electrodes 10 and 11. Consequently, the tissues LT1 are of the same sizes as each other, and hence can be treated at substantially the same temperatures. The tissue LT2 that is interposed between the tissues LT1 can be treated at a uniformly increased temperature by the heat conducted from the tissues LT1. Therefore, the treatment target tissue LT0 can be treated in its entirety in a well-balanced fashion.

### Embodiment 2

Next, Embodiment 2 of the disclosed technology will be described below:

The parts of Embodiment 2 which are identical to those of Embodiment 1 described hereinbefore are denoted by identical numeral references, and the description of those parts will be omitted or simplified.

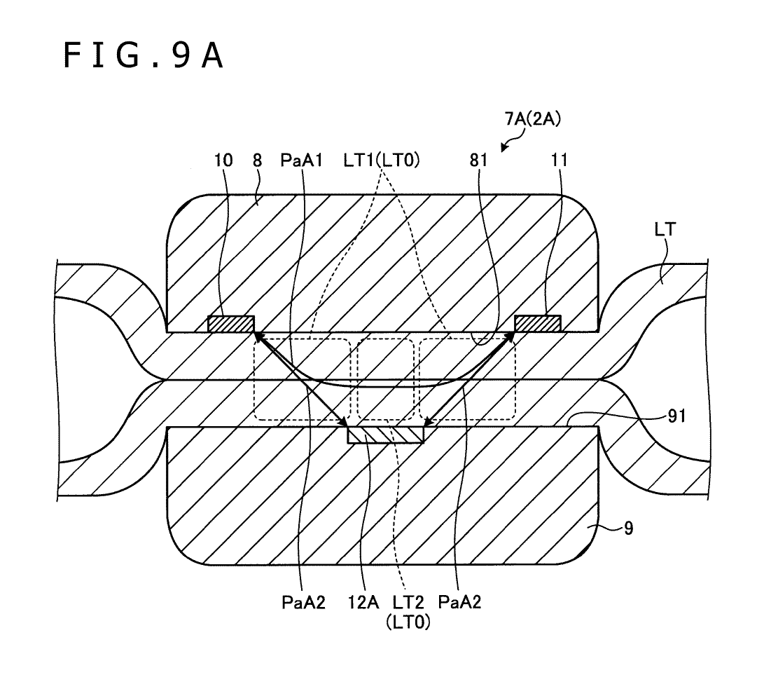

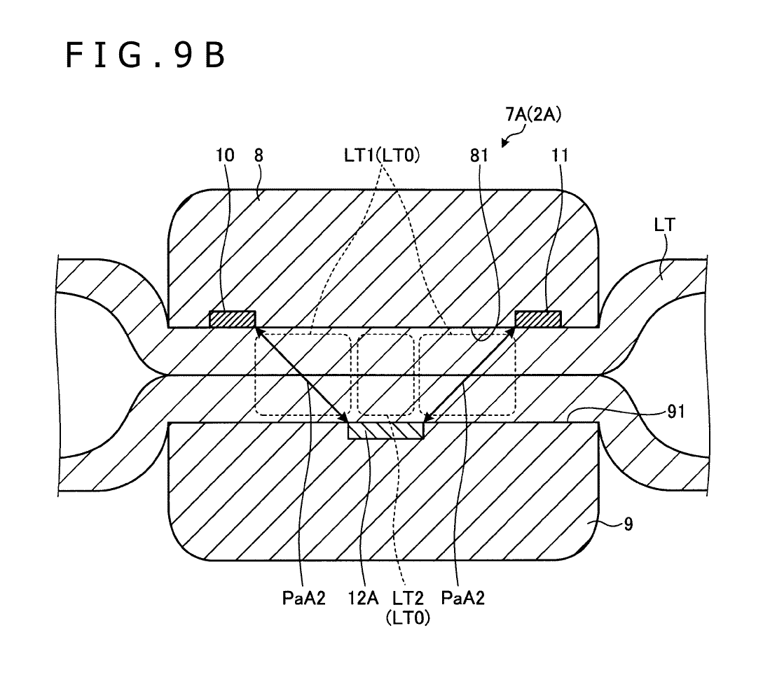

FIGS. 9A and 9B are views illustrating a grasper 7A of a treatment tool 2A according to Embodiment 2, and are cross-sectional views corresponding to FIG. 3. Specifically, FIG. 9A depicts a path for a high-frequency electric current in a former part of a treatment process, whereas FIG. 9B depicts a path for a high-frequency electric current in a latter part of the treatment process.

The treatment tool 2A according to Embodiment 2 incorporates a floating electrode 12A (FIGS. 9A and 9B), which is different from the floating electrode 12 of the treatment tool 2 according to Embodiment 1 described hereinbefore, only as to its material.

The floating electrode 12A according to Embodiment 2 is made of a material that is a nonconductor such as a resin or the like with an electrically conductive filler such as carbon, silver, or the like dispersed therein, e.g., an electrically conductive resin such as electrically conductive polyimide, electrically conductive PBI, electrically conductive PEEK, electrically conductive fluororubber, electrically conductive silicon, or the like. If the floating electrode 12A has a width of 1 mm, for example, then its volume resistivity should appropriately be in the range of approximately 0.1 to 10 Ω·m depending on which target region the living tissue LT is.

The electric resistance value of the tissue LT2 before being treated is 250 S2, for example. Furthermore, the electric resistance value of the tissue LT2 that is in a dry state, i.e., those water content is approximately 20%, is 800 S2, for example. In other words, according to Embodiment 2, the electric resistance value 500Ω of the floating electrode 12A is from a fraction up to substantially the same as, or close to but higher than, the electric resistance value of the tissue LT2 before being treated, and is lower than the electric resistance value of the tissue LT2 that is in the dry state.

Next, paths for high-frequency electric currents that flow when high-frequency electric power is supplied between the first and second electrodes 10 and 11 while the first and second grasping surfaces 81 and 91 are grasping the living tissue LT therebetween will be described below with reference to FIGS. 9A and 9B.

According to Embodiment 2, as described hereinbefore, the electric resistance value of the floating electrode 12A is from a fraction up to substantially the same as, or close to but higher than, the electric resistance value of the tissue LT2 before being treated. Therefore, in the former part of the treatment process, high-frequency electric currents flow between the first and second electrodes 10 and 11 along two paths PaA1 and PaA2, i.e., a path PaA1 that extends through only the treatment target tissue LT0, i.e., the tissues LT1 and LT2 and a path PaA2 that extends through both the tissues LT1 and the floating electrode 12A. The high-frequency electric current that flows along the path PaA1 generates Joule heat in the treatment target tissue LT0, whereas the high-frequency electric current that flows along the path PaA2 generates Joule heat in the tissues LT1.

The electric resistance value of the treatment target tissue LT0 goes higher as the treatment of the treatment target tissue LT0 progresses. As described hereinbefore, the electric resistance value of the floating electrode 12A is lower than the electric resistance value of the tissue LT2 in the dry state. In the latter part of the treatment process, therefore, as illustrated in FIG. 9B, much of the high-frequency electric current flows through the floating electrode 12A along the path PaA2. As the floating electrode 12A has a higher volume resistivity than the good conductor described in Embodiment 1, the high-frequency electric current that flows through the floating electrode 12A causes the floating electrode 12A to function as a tardy heat generator whose temperature rises owing to internal heat generation. In the latter part of the treatment process, therefore, the treatment target tissue LT0 is treated by being directly heated by the floating electrode 12A functioning as the tardy heat generator.

Embodiment 2 described hereinbefore offers the following advantages as well as the advantages that are the same as those of Embodiment 1:

With the treatment tool 2A according to Embodiment 2, the electric resistance value of the floating electrode 12A is from a fraction up to substantially the same as, or close to but higher than, the electric resistance value of the tissue LT2 before being treated, and is lower than the electric resistance value of the tissue LT2 that is in the dry state. Therefore, the treatment tool 2A can perform a treatment process in two stages as described hereinbefore. Specifically, in a first stage of treatment (FIG. 9A), the tissue LT2 can also be treated with Joule heat, making the treatment progress fast, compared with Embodiment 1. In a second stage of treatment (FIG. 9B), the direct heating by the floating electrode 12A functioning as the tardy heat generator can further make the treatment progress faster positively. With the conventional structure that is free of the floating electrode 12A, at the time the electric resistance value of the treatment target tissue LT0 has increased in excess of the voltage capacity of the power supply, for example, causing the power supply to fail to supply a high-frequency electric current, heating of the treatment target tissue LT0 cannot be induced. On the other hand, the floating electrode 12A allows the treatment to continue subsequent to the time referred to hereinbefore, making it possible to strengthen the treatment performance.

With the treatment tool 2A according to Embodiment 2, though the direct heating by the floating electrode 12A is a contributory factor, the region that is heated by the direct heating is limited within the first and second grasping jaws 8 and 9. Therefore, even though the direct heating by the floating electrode 12A is a contributory factor, the effect of heat on peripheral tissues that are positioned transversely outside of the first and second grasping jaws 8 and 9 in the periphery of the treatment target tissue LT0 is reduced, allowing the living tissue LT to be treated minimally invasively.

### Embodiment 3

Next, Embodiment 3 of the disclosed technology will be described below.

The parts of Embodiment 3 which are identical to those of Embodiment 1 described hereinbefore are denoted by identical numeral references, and the description of those parts will be omitted or simplified.

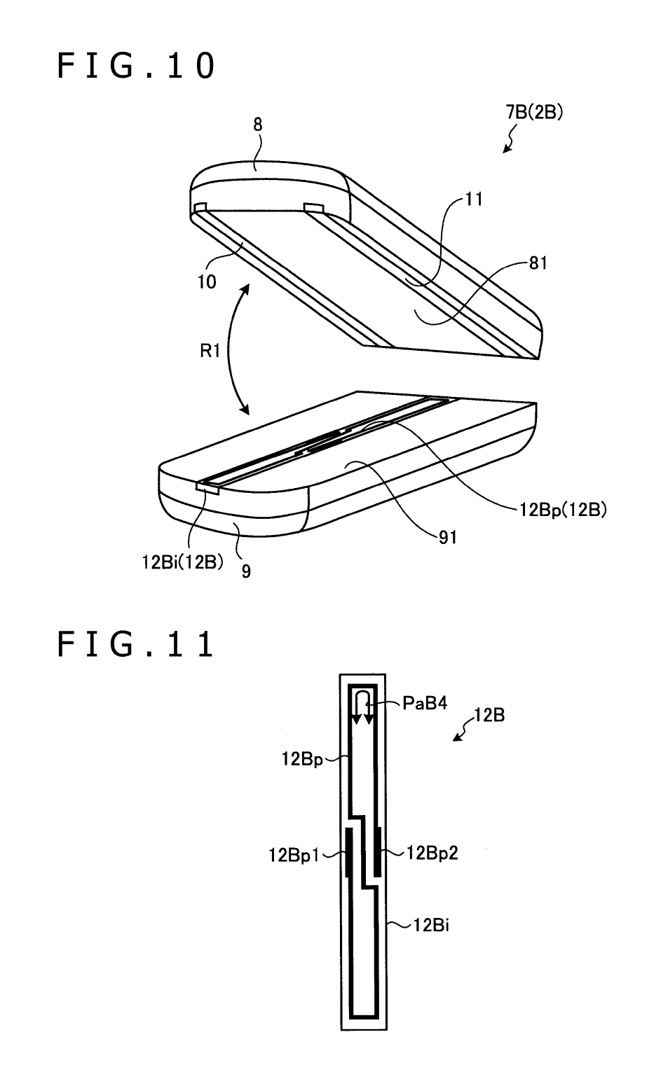

FIG. 10 is a view illustrating a grasper 7B of a treatment tool 2B according to Embodiment 3. Specifically, FIG. 10 is a perspective view corresponding to FIG. 2.

As illustrated in FIG. 10, the treatment tool 2B according to Embodiment 3 incorporates a floating electrode 12B, which is different from the floating electrode 12 of the treatment tool 2 (FIG. 2) according to Embodiment 1 described hereinbefore, only as to its material.

FIG. 11 is a view illustrating the floating electrode 12B. Specifically, FIG. 11 is a view of the floating electrode 12B as viewed from above along the direction normal to the second grasping surface 91.

As illustrated in FIG. 10 or 11, the floating electrode 12B according to Embodiment 3 includes a nonconductor 12Bi and a thin-film resistance pattern 12Bp.

The nonconductor 12Bi is made of ceramics such as aluminum nitride, alumina, or the like, or a resin such as polyimide or the like. The nonconductor 12Bi is of the same shape and size as the floating electrode 12 according to Embodiment 1 described hereinbefore.

The thin-film resistance pattern 12Bp is a portion corresponding to a thin-film resistance body according to the disclosed technology. The thin-film resistance pattern 12Bp is made of a good conductor such as Pt (Platinum), carbon, SUS (Stainless Steel), or the like, and is formed on an upper surface of the nonconductor 12Bi by evaporation, sputtering, or the like.

According to Embodiment 3, the thin-film resistance pattern 12Bp is constructed as one line. The thin-film resistance pattern 12Bp has pads 12Bp1 and 12Bp2 disposed on one and other ends thereof and facing each other widthwise. The thin-film resistance pattern 12Bp is substantially 8-shaped, extending from the one end, i.e., the pad 12Bp1, to the other end, i.e., the pad 12Bp2, along the outer edges of the upper surface of the nonconductor 12Bi. No wiring or the like is added for connection to the pads 12Bp1 and 12Bp2. Since it is not clear which longitudinal portions of the first and second grasping jaws 8 and 9 grasp the living tissue LT and what size those portions of the first and second grasping jaws 8 and 9 are during a surgical operation, the pads 12Bp1 and 12Bp2 are not required to be in the form of a substantially rectangular parallelepiped and to face each other widthwise. Instead, the pads 12Bp1 and 12Bp2 may have a conductor exposed at one transverse end and may also have a similar structure at the other transverse end. The conductor does not need to be exposed in its entirety, but may be covered with an insulative cover of polyimide or the like except openings defined respectively at the one and other transverse ends. At least one thin-film resistance body or a plurality of thin-film resistance bodies may be included which interconnect the conductors exposed through a pair of openings. A plurality of thin-film resistance bodies may be included which interconnect a plurality of pairs of conductors exposed through a plurality of pairs of openings. The electric resistance values of these thin-film resistance bodies should desirably be in the range of 50 to 500Ω.

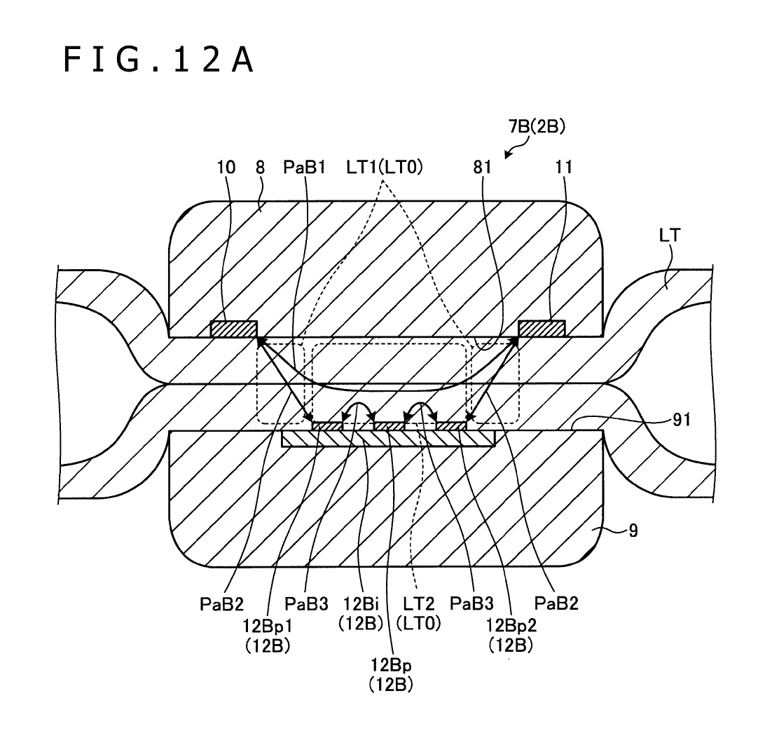

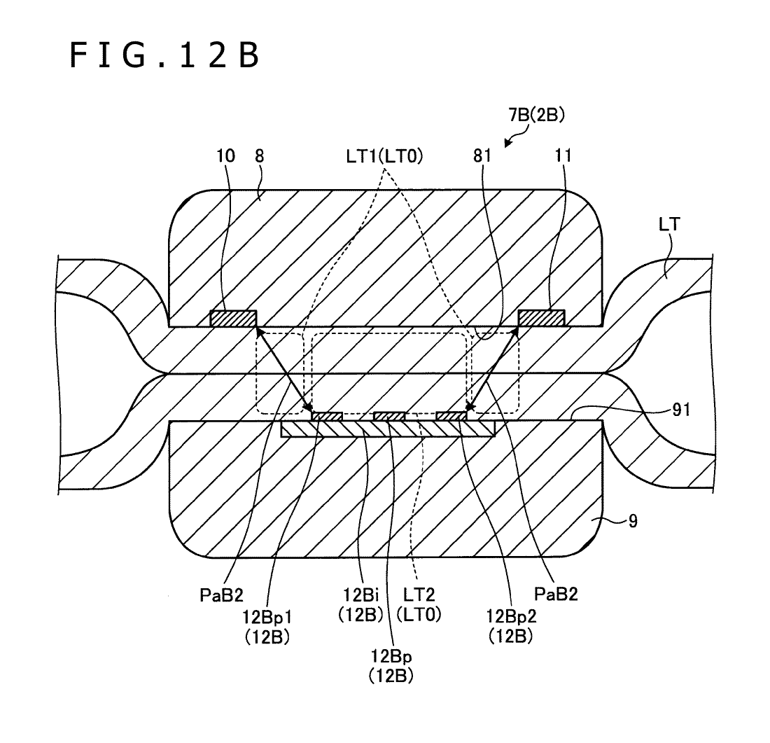

Paths for high-frequency electric currents that flow when high-frequency electric power is supplied between the first and second electrodes 10 and 11 while the first and second grasping surfaces 81 and 91 are grasping the living tissue LT therebetween will be described below with reference to FIGS. 12A and 12B.

FIGS. 12A and 12B are cross-sectional views corresponding to FIG. 3, and illustrate paths for high-frequency electric currents in former and latter parts of a treatment process.

According to Embodiment 3, as described hereinbefore, the electric resistance value of the floating electrode 12B is from a fraction up to substantially the same as, or close to but higher than, the electric resistance value of the tissue LT2 before being treated. In a former part of a treatment process, high-frequency electric currents flow between the first and second electrodes 10 and 11 along two paths PaB1 and PaB2, i.e., along a path PaB1 that extends through only the treatment target tissue LT0, i.e., the tissues LT1 and LT2, and a path PaB2 that extends through the tissues LT1 and the floating electrode 12B. The path PaB2 has a path PaB3 that extends through the tissue LT2, but not through the thin-film resistance pattern 12Bp, and a path PaB4 (FIG. 11) that extends through the thin-film resistance pattern 12Bp. In other words, the high-frequency electric currents that flow along the paths PaB1 and PaB2 generate Joule heat in the tissues LT1 and LT2, i.e., the treatment target tissue LT0.

As the treatment of the treatment target tissue LT0 progresses and the impedance of the tissue LT2 increases, the paths PaB1 and PaB3 become less likely to occur, but the paths PaB2 and PaB4 become essentially dominant. In other words, in a latter part of the treatment process, since the high-frequency electric current flows in the thin-film resistance pattern 12Bp along the path PaB4, the thin-film resistance pattern 12Bp functions as a tardy heat generator whose temperature rises owing to internal heat generation. Therefore, the treatment target tissue LT0 is treated by being directly heated by the floating electrode 12B functioning as the tardy heat generator.

Embodiment 3 described hereinbefore offers the following advantages as well as the advantages that are the same as those of Embodiment 2:

With the treatment tool 2B according to Embodiment 3, inasmuch as the resistance body that has had guaranteed reliability can be used without wiring, a heat-generating region can freely be configured by the shape and resistance density of the thin-film resistance pattern 12Bp. If a resistance body is used as a heater, then two wires are required for connection to the resistance body. Since such wires are not required, the second grasping jaw 9 can be reduced in size, i.e., the grasper 7B can be reduced in diameter.

### Embodiment 4

Embodiment 4 of the disclosed technology will be described below.

The parts of Embodiment 4 which are identical to those of Embodiment 1 described hereinbefore are denoted by identical numeral references, and the description of those parts will be omitted or simplified.

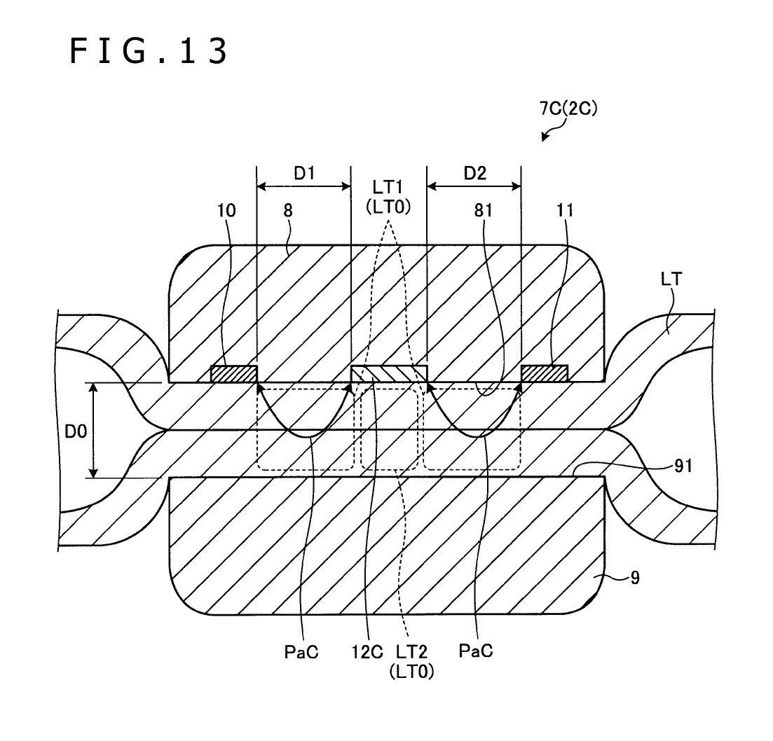

FIG. 13 is a view illustrating a grasper 7C of a treatment tool 2C according to Embodiment 4. Specifically, FIG. 13 is a cross-sectional view corresponding to FIG. 3.

As illustrated in FIG. 13, the treatment tool 2C according to Embodiment 4 is different from the treatment tool 2 (FIG. 3) according to Embodiment 1 described hereinbefore, as to the position where a floating electrode is disposed.

In the second grasping jaw 9 according to Embodiment 4, the second grasping surface 91 is free of the floating electrode 12, as illustrated in FIG. 13. Though the second grasping surface 91 according to Embodiment 4 is free of the floating electrode 12, the second grasping surface 91 has a flat shape as with Embodiment 1. The second grasping surface 91 may be coated with an electrically insulative coating material which is non-adhesive to living bodies as described hereinbefore in Embodiment 1.

In the first grasping jaw 8 according to Embodiment 4, the first grasping surface 81 includes a floating electrode 12C in addition to the first and second electrodes 10 and 11.

The floating electrode 12C is made of the same material as the floating electrode 12 described hereinbefore in Embodiment 1. The floating electrode 12C has the same shape, size, and function, i.e., the function as part of the path for the high-frequency electric current between the first and second electrodes 10 and 11, as the floating electrode 12.

The floating electrode 12C is embedded in an area of the first grasping surface 81 that is positioned centrally widthwise, and extends the entire length of the first grasping surface 81. The floating electrode 12C serves as part of the first grasping surface 81. The first grasping surface 81 according to Embodiment 4, though the floating electrode 12C is embedded therein, is shaped flatwise as with Embodiment 1 described hereinbefore. The lower surface of the floating electrode 12C as illustrated in FIG. 13 may be coated with an electrically conductive coating material which is non-adhesive to living bodies as described hereinbefore in Embodiment 1.

In Embodiment 4, the positional relationship of the first and second electrodes 10 and 11 and the floating electrode 12C as viewed along the directions in which the first and second grasping surfaces 81 and 91 face each other when the grasper 7C is in the closed state is the same as Embodiment 1. The spaced distance D1 between the first electrode 10 and the floating electrode 12C, i.e., the spaced distance D2 between the second electrode 11 and the floating electrode 12C, is set to be longer than the spaced distance D0 (FIG. 13).

The floating electrode 12C is not limited to the shape of the plate, but may be of a different shape such as a round rod or the like embedded such that it may have a protrusive portion that is small compared with the distance between the first and second grasping jaws 8 and 9. The floating electrode 12C may not necessarily be made of a bulk material, but may be made of a foil or thin film of a good conductor or an electrically conductive DLC thin film or the like that is formed by CVD or the like.

Next, a path for a high-frequency electric current that flows when high-frequency electric power is supplied between the first and second electrodes 10 and 11 while the first and second grasping surfaces 81 and 91 are grasping the living tissue LT therebetween will be described below with reference to FIG. 13.

The floating electrode 12C according to Embodiment 4 is made of a good conductor as with the floating electrode 12 described hereinbefore in Embodiment 1. Therefore, as illustrated in FIG. 13, a high-frequency electric current flows between the first and second electrodes 10 and 11 mainly along a path PaC that extends through the tissues LT1 and the floating electrode 12C. In other words, as with Embodiment 1, each of the tissues LT1 is treated by Joule heat. The tissue LT2 is treated by heat conduction from the Joule heat generated in each of the tissues LT1.

Embodiment 4 described hereinbefore offers the following advantages as well as the advantages that are the same as those of Embodiment 1:

With the treatment tool 2C according to Embodiment 4, the first grasping jaw 8 includes the first and second electrodes 10 and 11 and the floating electrode 12C. Stated otherwise, the second grasping jaw 9 does not have any of the first and second electrodes 10 and 11 and the floating electrode 12C. Therefore, the second grasping jaw 9 can be simplified in structure and can be reduced in size, i.e., the grasper 7C can be reduced in diameter.

With the treatment tool 2C according to Embodiment 4, the spaced distance D1 between the first electrode 10 and the floating electrode 12C, i.e., the spaced distance D2 between the second electrode 11 and the floating electrode 12C, is set to be longer than the spaced distance D0. If the spaced distance D1 or D2 is shorter than the spaced distance D0, then it is difficult for the path PaC for the high-frequency electric current to reach the interface between tissues to be joined, such as of a lumen, a blood vessel, or the like. However, as the spaced distance D1 or D2 is longer than the spaced distance D0, the path PaC for the high-frequency electric current can extend deeply thicknesswise to the tissue interface. Accordingly, the treatment can be effectively performed.

### Embodiment 5

Embodiment 5 of the disclosed technology will be described below.

The parts of Embodiment 5 which are identical to those of Embodiment 4 described hereinbefore are denoted by identical numeral references, and the description of those parts will be omitted or simplified.

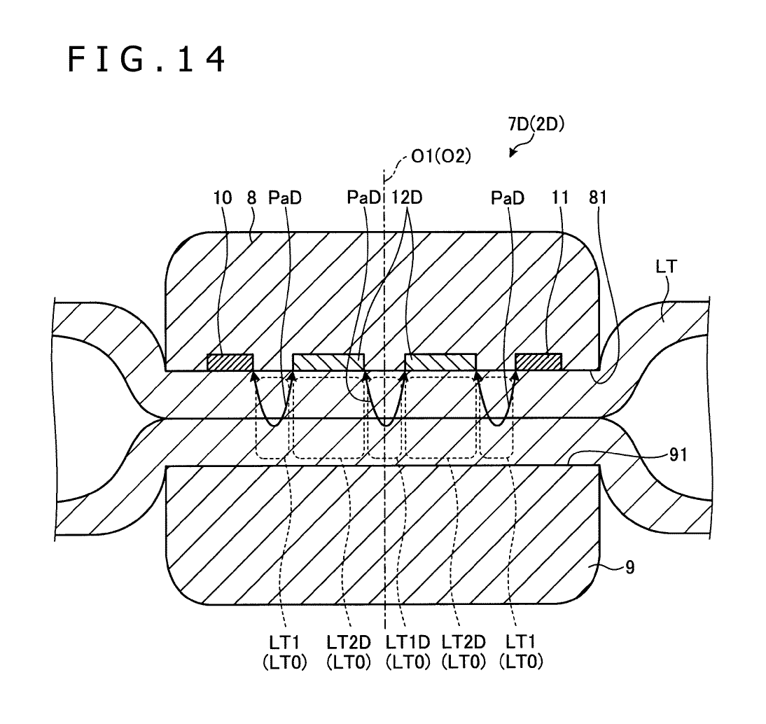

FIG. 14 is a view illustrating a grasper 7D of a treatment tool 2D according to Embodiment 5. Specifically, FIG. 14 is a cross-sectional view corresponding to FIG. 13.

As illustrated in FIG. 14, the treatment tool 2D according to Embodiment 5 is different from the treatment tool 2C (FIG. 13) according to Embodiment 4 described hereinbefore, as to the number of floating electrodes.

As illustrated in FIG. 14, the first grasping surface 81 according to Embodiment 5 has a plurality of, or two in Embodiment 5, floating electrodes 12D in addition to the first and second electrodes 10 and 11.

The two floating electrodes 12D are made of the same material as the floating electrode 12C described hereinbefore in Embodiment 4 and have the same shape, size, and function as the floating electrode 12C.

The floating electrodes 12D are embedded in respective areas of the first grasping surface 81 that is positioned between the first and second electrodes 10 and 11, and extends the entire length of the first grasping surface 81. More specifically, the floating electrodes 12D are disposed such that the distance between one of the floating electrodes 12D and the first electrode 10 adjacent thereto, the distance between the other floating electrode 12D and the second electrode 10 adjacent thereto, and the distance between the floating electrodes 12D are equal to each other. A transversely central position O1 between the two floating electrodes 12D is aligned with a transversely central position O2 between the first and second electrodes 10 and 11. These floating electrodes 12D serve as part of the first grasping surface 81. The first grasping surface 81 according to Embodiment 5, though the two floating electrodes 12D are embedded therein, is shaped flatwise as with Embodiment 4 described hereinbefore. The lower surfaces of the two floating electrodes 12D in the first grasping surface 81 as illustrated in FIG. 14 may be coated with an electrically conductive coating material which is non-adhesive to living bodies as described hereinbefore in Embodiment 4.

The number of the floating electrodes 12D is not limited to two, but may be three or more. Each of the floating electrodes 12D is not limited to the shape of the plate, but may be of a different shape such as a round rod or the like embedded such that it may have a protrusive portion that is small compared with the distance between the first and second grasping jaws 8 and 9. The floating electrodes 12D may not necessarily be made of a bulk material, but may be made of a foil or thin film of a good conductor or an electrically conductive DLC thin film or the like that is formed by CVD or the like.

Next, a path for a high-frequency electric current that flows when high-frequency electric power is supplied between the first and second electrodes 10 and 11 while the first and second grasping surfaces 81 and 91 are grasping the living tissue LT therebetween will be described below with reference to FIG. 14.

In the living tissue LT that is grasped by the first and second grasping surfaces 81 and 91, as viewed along the directions in which the first and second grasping surfaces 81 and 91 face each other, a tissue positioned between the two floating electrodes 12D will hereinafter be referred to as a tissue LT1D (FIG. 14), and tissues positioned between the tissues LT1 and LT1D as tissues LT2D (FIG. 14).

According to Embodiment 5, as described hereinbefore, the two floating electrodes 12D are uniformly spaced between the first and second electrodes 10 and 11. Therefore, when high-frequency electric power is supplied between the first and second electrodes 10 and 11, the two floating electrodes 12D are kept at uniformly assigned potentials between the potentials of the first and second electrodes 10 and 11. The two floating electrodes 12D are made of a good conductor as with the floating electrode 12C described hereinbefore in Embodiment 4. Therefore, as illustrated in FIG. 14, a high-frequency electric current flows between the first and second electrodes 10 and 11 mainly along a path PaD that extends through the tissues LT1 and LT1D and the floating electrode 12D. Thus, the tissue LT1D as well as the tissues LT1 is treated by Joule heat. The tissues LT2D are treated by heat conduction from the Joule heat generated in each of the tissues LT1 and LT1D. In other words, each of the tissues LT1, LT1D, and LT2D is a treatment target tissue LT0 to be treated.

Embodiment 5 described hereinbefore offers the following advantages as well as the advantages that are the same as those of Embodiment 4:

The treatment tool 2D according to Embodiment 5 has the two floating electrodes 12D. Therefore, the combined resistance between the first and second electrodes 10 and 11 can further be reduced. There are available more tissues LT1 where Joule heat is generated, i.e., more heat-generating spots, making it possible to treat the treatment target tissue LT0 more uniformly.

### Embodiment 6

Embodiment 6 of the disclosed technology will be described below.

The parts of Embodiment 6 which are identical to those of Embodiment 4 described hereinbefore are denoted by identical numeral references, and the description of those parts will be omitted or simplified.

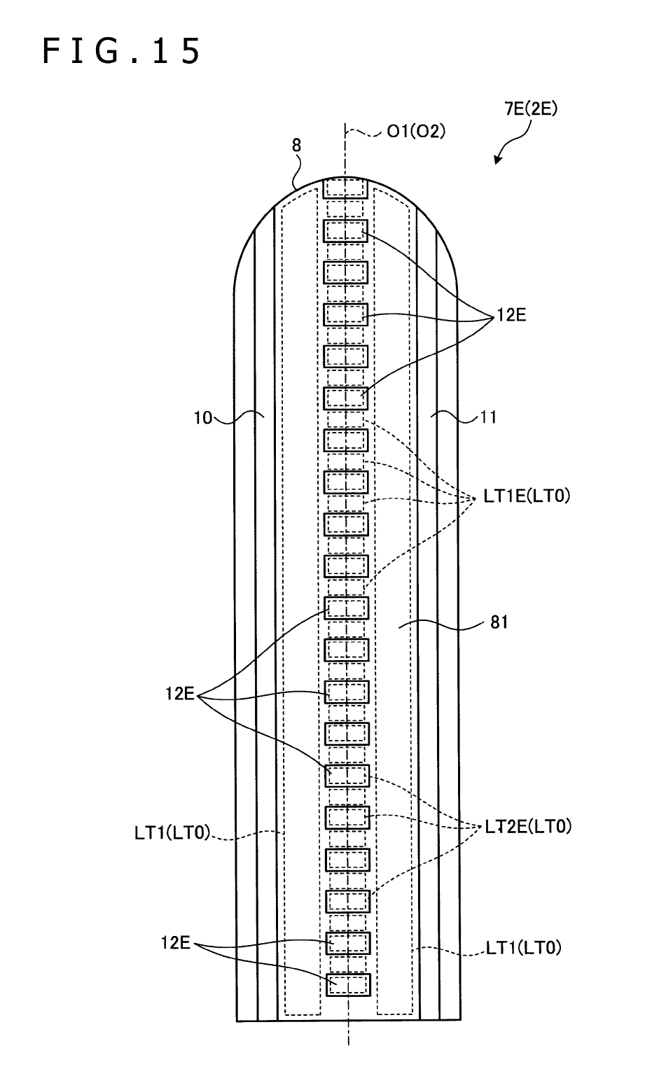

FIG. 15 is a view illustrating a grasper 7E of a treatment tool 2E according to Embodiment 6. Specifically, FIG. 15 is a view illustrating the first grasping surface 81 of the first grasping jaw 8.

As illustrated in FIG. 15, the treatment tool 2E according to Embodiment 6 is different from the treatment tool 2C (FIG. 13) according to Embodiment 4 described hereinbefore, as to the number of floating electrodes.

As illustrated in FIG. 15, the first grasping surface 81 according to Embodiment 6 has a plurality of, or twenty in Embodiment 5, floating electrodes 12E in addition to the first and second electrodes 10 and 11.

The twenty floating electrodes 12E are made of the same material as the floating electrode 12C described hereinbefore in Embodiment 4 and have the same shape, size, and function as the floating electrode 12C.

The floating electrodes 12E are identical in shape. Each of the floating electrodes 12E has a longitudinal dimension smaller than the floating electrode 12C described hereinbefore in Embodiment 4. The floating electrodes 12E are embedded in the first grasping surface 81 such that they are positioned between the first and second electrodes 10 and 11 and juxtaposed along the longitudinal directions. More specifically, each of the floating electrodes 12E has a transversely central position O1 that is aligned with a transversely central position O2 between the first and second electrodes 10 and 11. The floating electrodes 12E serve as part of the first grasping surface 81. The first grasping surface 81 according to Embodiment 6, though the floating electrodes 12E are embedded therein, is shaped flatwise as with Embodiment 4 described hereinbefore. The lower surfaces of the twenty floating electrodes 12E in the first grasping surface 81 as illustrated in FIG. 15 may be coated with an electrically conductive coating material which is non-adhesive to living bodies as described hereinbefore in Embodiment 4.

The number of the floating electrodes 12E is not limited to twenty, but may be any other number insofar as it is two or more. Each of the floating electrodes 12E is not limited to the plate, but may be of a different shape such as a round rod or the like embedded such that it may have a protrusive portion that is small compared with the distance between the first and second grasping jaws 8 and 9. The floating electrodes 12E may not necessarily be made of a bulk material, but may be made of a foil or thin film of a good conductor or an electrically conductive DLC thin film or the like that is formed by CVD or the like.

Next, paths for high-frequency electric current that flow when high-frequency electric power is supplied between the first and second electrodes 10 and 11 while the first and second grasping surfaces 81 and 91 are grasping the living tissue LT therebetween will be described below with reference to FIG. 15.

In the living tissue LT that is grasped by the first and second grasping surfaces 81 and 91, as viewed along the directions in which the first and second grasping surfaces 81 and 91 face each other, tissues positioned between the twenty floating electrodes 12E will hereinafter be referred to as tissues LT1E (FIG. 15), and tissues positioned between the tissues LT1E as tissues LT2E (FIG. 15).

According to Embodiment 6, there are a plurality of floating electrodes 12E and they are made of a good conductor, as with Embodiment 5 described hereinbefore. Therefore, as with Embodiment 5 described hereinbefore, between the first and second electrodes 10 and 11, a high-frequency electric current flows mainly between the first electrode 10 and the floating electrodes 12E, between the second electrode 11 and the floating electrodes 12E, and between the floating electrodes 12E. Thus, the tissue LT1E as well as the tissues LT1 are treated by Joule heat. The tissues LT2E are treated by heat conduction from the Joule heat generated in each of the tissues LT1 and LT1E. In other words, each of the tissues LT1, LT1E, and LT2E is a treatment target tissue LT0 to be treated.

Embodiment 6 described hereinbefore offers the following advantages as well as the advantages that are the same as those of Embodiment 5:

The treatment tool 2E according to Embodiment 6 has the twenty floating electrodes 12E juxtaposed along the longitudinal directions. Therefore, it is possible to make the intervals between the first and second electrodes 10 and 11 and the floating electrodes 12E wide, resulting in an electrically stable structure, compared with Embodiment 5 described hereinbefore.

The floating electrodes 12E are small discrete electrodes compared with Embodiments 1 and 4 according to which the floating electrodes 12 and 12C extend the entire length in the longitudinal directions. If the floating electrodes 12E are used as a tardy heat generator described hereinbefore in Embodiment 2, then they can avoid heat dissipation from themselves. On the other hand, heat is likely to dissipate from the larger floating electrodes 12 and 12C when used as a tardy heat generator.

Though the combined resistance between the first and second electrodes 10 and 11 is high compared with Embodiments 1 and 3 described hereinbefore, the combined resistance can be adjusted by using a material having a small volume resistivity as the floating electrodes 12E.

### Embodiment 7

Embodiment 7 of the disclosed technology will be described below.

The parts of Embodiment 7 which are identical to those of Embodiments 1 and 3 described hereinbefore are denoted by identical numeral references, and the description of those parts will be omitted or simplified.

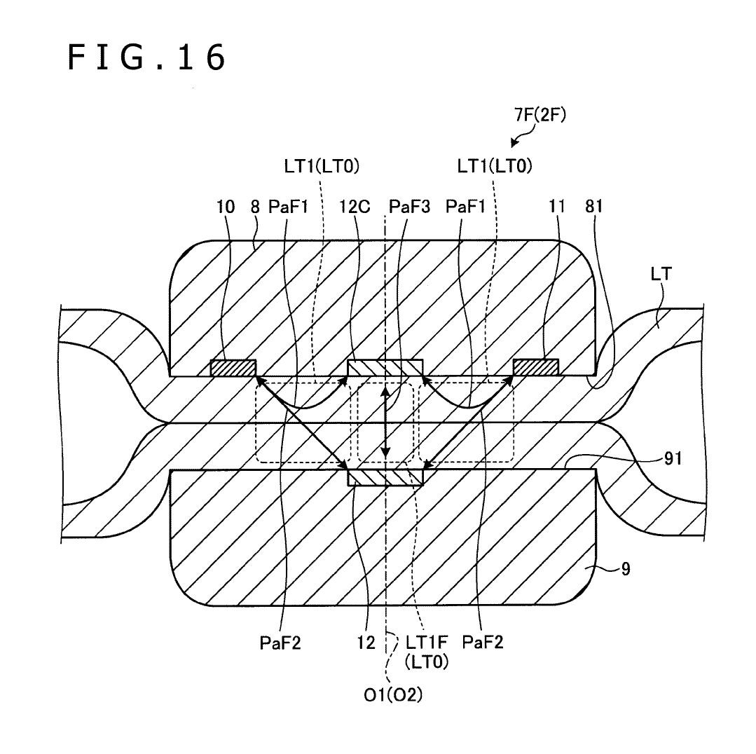

FIG. 16 is a view illustrating a grasper 7F of a treatment tool 2F according to Embodiment 7. Specifically, FIG. 16 is a cross-sectional view corresponding to FIGS. 3 and 13.

As illustrated in FIG. 16, the treatment tool 2F according to Embodiment 7 is different from the treatment tool 2 (FIG. 3) according to Embodiment 1 described hereinbefore and the treatment tool 2C (FIG. 13) according to Embodiment 4 described hereinbefore, as to the number of floating electrodes. Specifically, as illustrated in FIG. 16, the grasper 7F according to Embodiment 7 includes in combination the first grasping jaw 8 having the first and second electrodes 10 and 11 and the floating electrode 12C described hereinbefore in Embodiment 4 and the second grasping jaw 9 having the floating electrode 12 described hereinbefore in Embodiment 1.

Next, paths for high-frequency electric currents that flow when high-frequency electric power is supplied between the first and second electrodes 10 and 11 while the first and second grasping surfaces 81 and 91 are grasping the living tissue LT therebetween will be described below with reference to FIG. 16.

In the living tissue LT that is grasped by the first and second grasping surfaces 81 and 91, a tissue positioned between the two floating electrodes 12 and 12C will hereinafter be referred to as a tissue LT1F (FIG. 16).

According to Embodiment 7, there are two floating electrodes 12 and 12C and they are made of a good conductor, as with Embodiment 5 described hereinbefore. Therefore, as with Embodiment 5 described hereinbefore, between the first and second electrodes 10 and 11, high-frequency electric currents flow mainly between the first and second electrodes 10 and 11 and the floating electrode 12C, i.e., along a path PaF1, between the first and second electrodes 10 and 11 and the floating electrode 12, i.e., along a path PaF2, and between the floating electrodes 12 and 12C, i.e., along a path PaF3. Thus, the tissue LT1F as well as the tissues LT1 is treated by Joule heat. Each of the tissues LT1 and LT1F is a treatment target tissue LT0 to be treated.

The treatment tool 2F according to Embodiment 7 described hereinbefore offers the following advantages as well as the advantages that are the same as those of Embodiment 5:

With the treatment tool 2F according to Embodiment 7, the floating electrode 12C is disposed in the first grasping surface 81, whereas the floating electrode 12 is disposed in the second grasping surface 91. In each of the tissues LT1, Joule heat is generated on the first grasping surface 81 side by the high-frequency electric current flowing along the path PaF1, and Joule heat is generated on the second grasping surface 91 side by the high-frequency electric current flowing along the path PaF2. In other words, the tissues LT1 can be treated more uniformly. The tissue LT1F interposed between the tissues LT1 can be treated by Joule heat generated by the high-frequency electric current flowing along the path PaF3. Therefore, the progress of the treatment is made faster.

### Other Embodiments

The embodiments of the disclosed technology have been described hereinbefore. However, the disclosed technology should not be limited to Embodiments 1 through 7 described hereinbefore.

In Embodiments 1 through 7 described hereinbefore, the first grasping jaw 8 is disposed upwardly of the second grasping jaw 9. However, the disclosed technology is not limited to such a structure. Instead, the first grasping jaw 8 may be disposed downwardly of the second grasping jaw 9. The shaft 6 or the grasper 7, i.e., 7A through 7F, may be made rotatable about the central axis of the shaft 6 with respect to the handle 5.

In Embodiments 1 through 7 described hereinbefore, the first and second grasping surfaces 81 and 91 are flat surfaces. The disclosed technology is not limited to such a structure. Instead, the first and second grasping surfaces 81 and 91 may be shaped otherwise for the purpose of increasing the treatment performance. For example, one of the first and second grasping surfaces 81 and 91 may be of a flat shape, whereas the other may be of a protrusion shape. Alternatively, one of the first and second grasping surfaces 81 and 91 may be of a protrusion shape, whereas the other may be of a recess shape. For effectively making an incision in the living tissue LT as a treatment process, at least one of the first and second grasping surfaces 81 and 91 may have a portion having a V-shaped cross section at the incising position in the vicinity of the other grasping surface.

In Embodiments 1 through 7 described hereinbefore, the two electrodes, i.e., the first and second electrodes 10 and 11, are employed for imparting high-frequency energy. However, the number of such electrodes is not limited to two, but may be three or more.

In Embodiments 1 through 7 described hereinbefore, the positions where the first and second electrodes 10 and 11 and the floating electrode 12, i.e., 12A through 12E, are not limited to the positions described hereinbefore in Embodiments 1 through 7. Insofar as the floating electrode 12, i.e., 12A through 12E, is disposed between the first and second electrodes 10 and 11 as viewed along the directions in which the first and second grasping surfaces 81 and 91 face each other when the grasper is in the closed state, the electrodes may be disposed in other positions. For example, while the first and second electrodes 10 and 11 are disposed in the first grasping surface 81, i.e., in one grasping surface, according to Embodiments 1 through 7 described hereinbefore, the first and second electrodes 10 and 11 may be disposed in different grasping surfaces, respectively.

In Embodiments 1 through 7 described hereinbefore, the treatment tool 2, i.e., 2A through 2F, treats the living tissue LT by imparting high-frequency energy thereto. The disclosed technology is not limited to such a process. Instead, the treatment tool 2 may treat the living tissue LT by imparting thermal energy, ultrasonic energy, or optical energy such as laser or the like, other than high-frequency energy, to the living tissue LT.

In Embodiments 4 through 7 described hereinbefore, the floating electrodes 12C through 12E are made of a good conductor. However, they are not limited to such a material. Instead, as with the floating electrode 12A described hereinbefore in Embodiment 2 and the floating electrode 12B described hereinbefore in Embodiment 3, the floating electrodes 12C through 12E may be made of an electrically conductive resin or a nonconductor and a thin-film resistance pattern, thereby making themselves into a tardy heat generator.

In sum, one aspect of the disclosed technology is directed to a treatment tool comprises a first grasping jaw having a first grasping surface. A second grasping jaw having a second grasping surface and is configured to engage with the first grasping jaw so as to relatively pivot with respect to one another for holding a living tissue therebetween. A first electrode is disposed on the first grasping surface. A second electrode is disposed on either the first grasping surface or the second grasping surface and is configured to generate high-frequency energy in tandem with the first electrode to the living tissue held therebetween. A floating electrode is disposed in at least one of the first grasping surface and the second grasping surface. The floating electrode having a first end and a second end. Both of the first end and second end is disposed between the first electrode and the second electrode as viewed along directions in which the first grasping surface and the second grasping surface face each other when the first grasping surface and the second grasping surface face each other.

The floating electrode has an electric resistance value lower than the electric resistance value of the living tissue. The floating electrode has an electric resistance value lower than the electric resistance value of the living tissue in a dry state. The floating electrode has at least one electrically exposed area on one end thereof on a first electrode side and an opposed end thereof on a second electrode side. The floating electrode including at least one thin-film resistance body interconnecting the area on the one end and the area on the opposed end. The second electrode and the floating electrode are disposed in the first grasping surface. Each of spaced distance between the first electrode and the floating electrode and spaced distance between the second electrode and the floating electrode is longer than spaced distance between the first grasping surface and the second grasping surface when the first grasping surface and the second grasping surface hold the living tissue therebetween. The floating electrode has a length longer than spaced distance between the first grasping surface and the second grasping surface as viewed along longitudinal directions of the first grasping surface and the second grasping surface when the first grasping surface and the second grasping surface are in contact with one another. The floating electrode is defined by a plurality of the floating electrodes. The plurality of floating electrodes are disposed in one of the first grasping surface and the second grasping surface or both of the respective first and second grasping surfaces. The plurality of floating electrodes are disposed in each of the first grasping surface and the second grasping surface. The floating electrode has a central position aligned with a central position between the first electrode and the second electrode as viewed along the directions in which the first grasping surface and the second grasping surface face each other when the first grasping surface and the second grasping surface face each other.

Another aspect of the disclosed technology is directed to a treatment system used for treatment of a body tissue by applying electrical energy thereto. The treatment system comprises a controller and a treatment tool configured to be attached to controller. The treatment tool comprises a shaft having a first end and a second end. A handle is attached to the first end. Respective first and second grasping jaws each of which having respective first and second grasping surfaces configured to be engaged with the second end of the shaft so as to pivot with respect to one another for holding living tissue therebetween during the treatment. A first electrode is disposed on the first grasping surface. A second electrode is disposed on either the first grasping surface or the second grasping surface and is configured to generate high-frequency energy in tandem with the first electrode to the living tissue being held therebetween. At least one floating electrode is disposed in at least one of the respective first and second grasping surfaces so that the treatment tool being capable of reducing a voltage required to treat the body tissue while performing the treatment without reducing a size of the body tissue.

The floating electrode has an electric resistance value lower than the electric resistance value of the living tissue. The floating electrode becomes part of a path of high-frequency electric current when body tissue is grasped by the respective first and grasping jaws so as to reduce resistance between the respective first and second electrodes. The floating electrode is electrically communicating with the respective first and second electrodes without being connected to the controller. The floating electrode is defined by a plurality of the floating electrodes.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent.

Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

### NUMERAL REFERENCE LIST

1 Treatment system
2, 2A to 2F Treatment tool
3 Controller
4 Foot switch
5 Handle
6 Shaft
7, 7A to 7F Grasper
8, 9 First, second grasping jaw
10, 11 First, second electrode
12, 12A to 12E Floating electrode
12Bi Nonconductor
12Bp Thin-film resistance pattern
12Bp1, 12Bp2 Pad
51 Manipulating knob
81, 91 First, second grasping surface
C Electric cable
D0 to D2 Spaced distance
LT Living tissue
LT0 Treatment target tissue
LT1, LT1D to LT1f, LT2, LT2D, LT2E Tissue
O1, O2 Central position
Pa, PaA1, PaA2, PaB1 to PaB4, PaC, PaD, PaF1 to PaF3 Path
R1 Arrow
W1 Length

What is claimed is:

1. A treatment tool comprising:
a first grasping jaw having a first grasping surface;
a second grasping jaw having a second grasping surface and being configured to engage with the first grasping jaw so as to relatively pivot with respect to one another for holding a living tissue therebetween;
a first electrode being disposed on the first grasping surface;
a second electrode being disposed on either the first grasping surface or the second grasping surface and configured to generate high-frequency energy in tandem with the first electrode to the living tissue held therebetween; and
a floating electrode disposed in at least one of the first grasping surface and the second grasping surface, the floating electrode having a first end and a second end, both of the first end and second end being disposed between the first electrode and the second electrode as viewed along directions in which the first grasping surface and the second grasping surface face each other when the first grasping surface and the second grasping surface face each other.

2. The treatment tool of claim 1, wherein the floating electrode has an electric resistance value lower than the electric resistance value of the living tissue.

3. The treatment tool of claim 2, wherein the floating electrode has an electric resistance value lower than the electric resistance value of the living tissue in a dry state.

4. The treatment tool of claim 2, wherein the floating electrode has at least one electrically exposed area on one end thereof on a first electrode side and an opposed end thereof on a second electrode side, the floating electrode including at least one thin-film resistance body interconnecting the area on the one end and the area on the opposed end.

5. The treatment tool of claim 1, wherein the second electrode and the floating electrode are disposed in the first grasping surface.

6. The treatment tool of claim 5, wherein each of spaced distance between the first electrode and the floating electrode and spaced distance between the second electrode and the floating electrode is longer than spaced distance between the first grasping surface and the second grasping surface when the first grasping surface and the second grasping surface hold the living tissue therebetween.

7. The treatment tool of claim 1, wherein the floating electrode has a length longer than spaced distance between the first grasping surface and the second grasping surface as viewed along longitudinal directions of the first grasping surface and the second grasping surface when the first grasping surface and the second grasping surface are in contact with one another.

8. The treatment tool of claim 1, wherein the floating electrode is defined by a plurality of the floating electrodes.

9. The treatment tool of claim 8, wherein the plurality of floating electrodes are disposed in one of the first grasping surface and the second grasping surface or both of the respective first and second grasping surfaces.

10. The treatment tool of claim 8, wherein the plurality of floating electrodes are disposed in each of the first grasping surface and the second grasping surface.

11. The treatment tool of claim 1, wherein the floating electrode has a central position aligned with a central position between the first electrode and the second electrode as viewed along the directions in which the first grasping surface and the second grasping surface face each other when the first grasping surface and the second grasping surface face each other.

12. A treatment system used for treatment of a body tissue by applying electrical energy thereto, the treatment system comprising:
a controller; and
a treatment tool configured to be attached to controller, the treatment tool comprising
a shaft having a first end and a second end,
a handle being attached to the first end,
respective first and second grasping jaws each of which having respective first and second grasping surfaces configured to be engaged with the second end of the shaft so as to pivot with respect to one another for holding living tissue therebetween during the treatment,
a first electrode being disposed on the first grasping surface;
a second electrode being disposed on either the first grasping surface or the second grasping surface and configured to generate high-frequency energy in tandem with the first electrode to the living tissue being held therebetween, and
at least one floating electrode disposed in at least one of the respective first and second grasping surfaces so that the treatment tool being capable of reducing a voltage required to treat the body tissue while performing the treatment without reducing a size of the body tissue.

13. The treatment system of claim 12, wherein the floating electrode has an electric resistance value lower than the electric resistance value of the living tissue.

14. The treatment system of claim 12, wherein the floating electrode becomes part of a path of high-frequency electric current when body tissue is grasped by the respective first and grasping jaws so as to reduce resistance between the respective first and second electrodes.

15. The treatment system of claim 12, wherein the floating electrode is electrically communicating with the respective first and second electrodes without being connected to the controller.

16. The treatment system of claim 12, wherein the floating electrode is defined by a plurality of the floating electrodes.
